(19) United States
(12) Patent Application Publication
(10) Pub. No.: US 2007/0249635 A1
(43) Pub. Date: Oct. 25, 2007

(54) Fungicidal Mixtures Based on a Triazolopyrimidine Derivative and a Conazole

(75) Inventors: Jordi Tormo i Blasco, Weinheim (DE); Thomas Grote, Wachenheim (DE); Maria Scherer, Godramstein (DE); Reinhard Stierl, Kaohsiung County (TW); Siegfried Strathmann, Limburgerhof (DE); Ulrich Schofl, Bruhl (DE)

Correspondence Address: BIRCH STEWART KOLASCH & BIRCH, PO BOX 747, FALLS CHURCH, VA, 22040-0747, US

(73) Assignee: BASF Aktiengesellschaft, Ludwigshafen (DE)

(21) Appl. No.: 10/579,140

(22) PCT Filed: Nov. 18, 2004
(86) PCT No.: PCT/EP04/13068

§ 371 Date: Mar. 19, 2007

(30) Foreign Application Priority Data
Nov. 27, 2003 (DE) 10356105.6

(51) Int. Cl. A61K 31/519; A61K 31/41

(52) U.S. Cl. 514/259.31; 514/383

(57) ABSTRACT

Fungicidal mixtures comprising, as active components,
1) the triazolopyrimidine derivative of the formula I 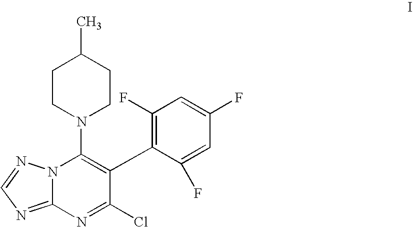 and
2) cyproconazole of the formula II, 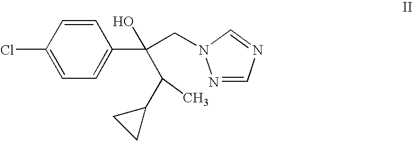 in a synergistically effective amount, methods for controlling harmful fungi from the class of the Oomycetes using mixtures of the compound I with the compound II, the use of the compound I with the compound II for preparing such mixtures and compositions comprising these mixtures are described.

The present invention relates to fungicidal mixtures comprising, as active components,
1) the triazolopyrimidine derivative of the formula I 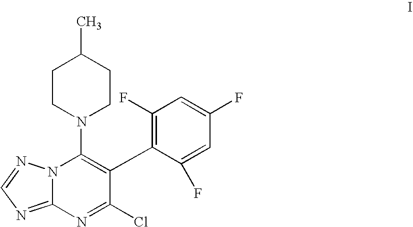
   and
2) cyproconazole of the formula II, 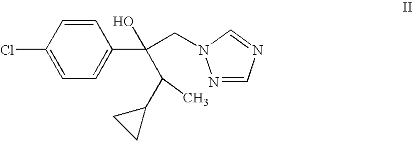
   in a synergistically effective amount.

Moreover, the invention relates to a method for controlling harmful fungi from the class of the Oomycetes using mixtures of the compound I with the compound II, to the use of the compound I with the compound II for preparing such mixtures and to compositions comprising these mixtures.

The compound I, 5-chloro-7-(4-methylpiperidin-1-yl)-6-(2,4,6-trifluorophenyl)-[1,2,4]triazolo[1,5-a]pyrimidine, its preparation and its action against harmful fungi are known from the literature (WO 98/46607).

The compound II 2-(4-chlorophenyl)-3-cyclopropyl- 1-[1,2,4]triazol-1 -ylbutan-2-ol, its preparation and its action against harmful fungi are also known from the literature (U.S. Pat. No. 4,664,696; common name: cyproconazole).

Mixtures of triazolopyrimidine derivatives with cyproconazole are known in a general manner from EP-A 988 790. The compound I is embraced by the general disclosure of this publication, but not explicitly mentioned. Accordingly, the combination of compound I with cyproconazole is novel.

The synergistic mixtures of triazolopyrimidines described in EP-A 988 790 are described as being fungicidally active against various diseases of cereals, fruit and vegetables, in particular mildew on wheat and barley or gray mold on apples. However, the fungicidal action of these mixtures against harmful fungi from the class of the Oomycetes is unsatisfactory.

The biological behavior of Oomycetes is clearly different from that of the Ascomycetes, Deuteromycetes and Basidiomycetes, since Oomycetes are biologically more closely related to algae than to fungi. Accordingly, what is known about the fungicidal activity of active compounds against “true fungi” such as Ascomycetes, Deuteromycetes and Basidiomycetes can be applied only to a very limited extent to Oomycetes.

Oomycetes cause economically relevant damage to various crop plants. In many regions, infections by Phytophthora infestans in the cultivation of potatoes and tomatoes are the most significant plant diseases. In viticulture, considerable damage is caused by peronospora of grapevines.

There is a constant demand for novel compositions against Oomycetes in agriculture, since there is already widespread resistance of the harmful fungi to the products established in the market, such as, for example, metalaxyl and active compounds of a similar structure.

Practical agricultural experience has shown that the repeated and exclusive application of an individual active compound in the control of harmful fungi leads in many cases to a rapid selection of such fungus strains which have developed natural or adapted resistance against the active compound in question. Effective control of these fungi with the active compound in question is then no longer possible.

To reduce the risk of selection of resistant fungus strains, mixtures of different active compounds are nowadays preferably employed for controlling harmful fungi. By combining active compounds having different mechanisms of action, it is possible to ensure a successful control over a relatively long period of time.

It is an object of the present invention to provide, with a view to effective resistance management and an effective control of harmful fungi from the class of the Oomycetes at application rates which are as low as possible, mixtures which, at a total amount of active compounds applied which is as low as possible, have a satisfactory effect against the harmful fungi.

We have found that this object is achieved by the mixtures defined at the outset. Moreover, we have found that simultaneous, that is joint or separate, application of the compound I and the compound II or successive application of the compounds I and the compound II allows better control of Oomycetes than is possible with the individual compounds (synergistic mixtures).

In addition, the combination according to the invention of the compounds I and II is also suitable for controlling other pathogens such as., for example, Septoria and Puccinia species in cereals and Alternaria and Boytritis species in vegetables, fruit and grapevines.

They can also be used in the protection of materials (e.g. the protection of wood), for example against Paecilomyces variotii.

When preparing the mixtures, it is preferred to employ the pure active compounds I and II to which further active compounds against harmful fungi or against other pests, such as insects, arachnids or nematodes, or else herbicidal or growth-regulating active compounds or fertilizers can be added according to need.

Other suitable active compounds in the above sense are in particular active compounds selected from the following groups:
acylalanines, such as benalaxyl, ofurace, oxadixyl,
amine derivates, such as aldimorph, dodemorph, fenpropidin, guazatine, iminoctadine, tridemorph,
anilinopyrimidines, such as pyrimethanil, mepanipyrim or cyprodinil,
antibiotics, such as cycloheximid, griseofulvin, kasugamycin, natamycin, polyoxin or streptomycin,
azoles, such as bitertanol, bromoconazole, cyproconazole, difenoconazole, dinitroconazole, enilconazole, fenbuconazole, fluquinconazole, flusilazole, flutriafol, hexaconazole, imazalil, ipconazole, myclobutanil, penconazole, propiconazole, prochloraz, prothioconazole, simeconazole, tetraconazole, triadimefon, triadimenol, triflumizole, triticonazole,
dicarboximides, such as myclozolin, procymidone,
dithiocarbamates, such as ferbam, nabam, metam, propineb, polycarbamate, ziram, zineb,
heterocyclic compounds, such as anilazine, boscalid, oxycarboxin, cyazofamid, dazomet, famoxadone, fenamidone, fuberidazole, flutolanil, furametpyr, isoprothiolan, mepronil, nuarimol, probenazole, pyroquilon, silthiofam, thiabendazole, thifluzamide, tiadinil, tricyclazole, triforine,
nitrophenyl derivatives, such as binapacryl, dinocap, dinobuton, nitrophthalisopropyl,
other fungicides, such as acibenzolar-S-methyl, carpropamid, chlorothalonil, cyflufenamid, cymoxanil, diclomezin, diclocymet, diethofencarb, edifenphos, ethaboxam, fentin acetate, fenoxanil, ferimzone, fosetyl, hexachlorobenzene, metrafenon, pencycuron, propamocarb, phthalide, toloclofos-methyl, quintozene, zoxamid,
strobilurins, such as fluoxastrobin, metominostrobin, orysastrobin or piraclostrobin,
sulfenic acid derivatives, such as captafol,
cinnamides and analogous compounds, such as flumetover.

In one embodiment of the mixtures according to the invention, a further fungicide III or two fungicides III and IV are added to the compounds I and II. Preference is given to mixtures of the compounds I and II and a component III. Particular preference is given to mixtures of the compounds I and II.

The mixtures of the compound I and the compound II or the simultaneous, that is joint or separate, use of the compound I and the compound II are distinguished by being very highly active against phytopathogenic fungi from the class of the Oomycetes, in particular of Phytophthora infestans on potatoes and tomatoes and Plasmopara viticola on grapevines. They can be used in crop protection as foliar— and soil-acting fungicides.

They are particularly important for controlling Oomycetes on various crop plants such as vegetable plants (for example cucumbers, beans and cucurbits), potatoes, tomatoes, grapevines and the corresponding seeds.

They are particularly suitable for controlling late blight on tomatoes and potatoes caused by Phytophthora infestans and downy mildew of grapevines (peronospora of grapevines) caused by Plasmopara viticola.

The compound I and the compound II can be applied simultaneously, that is jointly or separately, or in succession, the sequence, in the case of separate application, generally not having any effect on the result of the control measures.

The compound I and the compound II are usually applied in a weight ratio of from 100:1 to 1:100, preferably from 20:1 to 1:20, in particular from 10:1 to 1:10.

The components III and, if appropriate, IV are added, if desired to the compound I in a ratio of from 20:1 to 1:20.

Depending on the type of compound and the desired effect, the application rates of the mixtures according to the invention are from 5 g/ha to 1000 g/ha, preferably from 50 to 900 g/ha, in particular from 50 to 750 g/ha.

Correspondingly, the application rates for the compound I are generally from 1 to 1000 g/ha, preferably from 10 to 900 g/ha, in particular from 20 to 750 g/ha.

Correspondingly, the application rates for the compound II are generally from 1 to 1000 g/ha, preferably from 10 to 500 g/ha, in particular from 40 to 100 g/ha.

In the treatment of seed, application rates of mixture are generally from 1 to 1000 g/100 kg of seed, preferably from 1 to 200 g/100 kg, in particular from 5 to 100 g/100 kg.

The method for controlling harmful fungi is carried out by the separate or joint application of the compound I and the compound II or of the mixtures of the compound I and the compound II by spraying or dusting the seeds, the plants or the soils before or after sowing of the plants or before or after emergence of the plants. The compounds are preferably applied by spraying the leaves.

The mixtures according to the invention, or the compounds I and II, can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner, for example by extending the active compound with solvents and/or carriers, if desired using emulsifiers and dispersants.

Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignin-sulfite waste liquors and methylcellulose.

Suitable surfactants used are alkali metal, alkaline earth metal and ammonium salts of lignosulfonic acid, naphthalenesulfonic acid, phenolsulfonic acid, dibutyinaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone or water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compounds. In this case, the active compounds are employed in a purity of from 90% to 100%, preferably 95% to 100% (according to NMR spectrum).

The following are examples of formulations: 1. Products for dilution with water
A) Water-soluble concentrates (SL)

10 parts by weight of the active compounds are dissolved in water or in a water-soluble solvent. As an alternative, wetters or other auxiliaries are added. The active compound dissolves upon dilution with water.
B) Dispersible concentrates (DC)

20 parts by weight of the active compounds are dissolved in cyclohexanone with addition of a dispersant, for example polyvinylpyrrolidone. Dilution with water gives a dispersion.
C) Emulsifiable concentrates (EC)

15 parts by weight of the active compounds are dissolved in xylene with addition of calcium dodecylbenzenesulfonate and castor oil ethoxylate (in each case 5% strength). Dilution with water gives an emulsion.
D) Emulsions (EW, EO)

40 parts by weight of the active compounds are dissolved in xylene with addition of calcium dodecylbenzenesulfonate and castor oil ethoxylate (in each case 5% strength). This mixture is introduced into water by means of an emulsifier machine (Ultraturrax) and made into a homogeneous emulsion. Dilution with water gives an emulsion.
E) Suspensions (SC, OD)

In an agitated ball mill, 20 parts by weight of the active compounds are comminuted with addition of dispersants, wetters and water or an organic solvent to give a fine active compound suspension. Dilution with water gives a stable suspension of the active compound.
F) Water-dispersible granules and water-soluble granules (WG, SG)

50 parts by weight of the active compounds are ground finely with addition of. dispersants and wetters and made as water-dispersible or water-soluble granules by means of technical appliances (for example extrusion, spray tower, fluidized bed). Dilution with water gives a stable dispersion or solution of the active compound.
G) Water-dispersible powders and water-soluble powders (WP, SP)

75 parts by weight of the active compounds are ground in a rotor-stator mill with addition of dispersants, wetters and silica gel. Dilution with water gives a stable dispersion or solution of the active compound.

2. Products to be applied undiluted
H) Dustable powders (DP) 5 parts by weight of the active compounds are ground finely and mixed intimately with 95% of finely divided kaolin. This gives a dustable product.
I) Granules (GR, FG, GG, MG)

0.5 part by weight of the active compounds is ground finely and associated with 95.5% carriers. Current methods are extrusion, spray-drying or the fluidized bed. This gives granules to be applied undiluted.
J) ULV solutions (UL)

10 parts by weight of the active compounds are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), it being possible to apply formulations comprising over 95% by weight of active compound, or even to apply the active compound without additives.

Oils of various types, wetters, adjuvants, herbicides, fungicides, other pesticides, or bactericides may be-added to the active compounds, even, if appropriate, just immediately prior to use (tank mix). These agents can be admixed with the compositions according to the invention in a weight ratio from 1:10 to 10:1.

The compounds I and II, the mixtures or the corresponding formulations are applied by treating the harmful fungi, the plants, seeds, soils, areas, materials or spaces to be kept free from them with a fungicidally effective amount of the mixture or, in the case of separate application, of the compounds I and II. Application can be carried out before or after infection by the harmful fungi.

The fungicidal action of the compound and of the mixtures can be demonstrated by the following experiments:

The active compounds, separately or jointly, were prepared as a stock solution comprising 0.25% by weight of active compound in acetone or DMSO. 1 % by weight of the emulsifier Uniperol® EL (wetting agent having emulsifying and dispersant action based on ethoxylated alkylphenols) was added to this solution, and the mixture was diluted with water to the desired concentration.

Use example—activity against peronospora of gravevines caused by Plasmopara viticola

Leaves of potted vines of the cultivar “Riesling” were sprayed to runoff point with an aqueous suspension having the concentration of active compound stated below. The next day, the undersides of the leaves were inoculated with an aqueous zoospore suspension of Plasmopara viticola. The grapevines were then initially placed in a water-vapor-saturated chamber at 24° C. for 48 hours and then in a greenhouse at 20-30° C. for 5 days. After this period of time, the plants were again placed in a humid chamber for 16 hours to promote sporangiophore eruption. The extent of the development of the disease on the undersides of the leaves was then determined visually.

The visually determined percentages of infected leaf areas were converted into efficacies as % of the untreated control:

The efficacy (E) is calculated as follows using Abbot's formula:
E=(1−α/β)·100
α corresponds to the fungicidal infection of the treated plants in % and
β corresponds to the fungicidal infection of the untreated (control) plants in %

An efficacy of 0 means that the infection level of the treated plants corresponds to that of the untreated control plants; an efficacy of 100 means that the treated plants were not infected.

The expected efficacies of combinations of active compounds were determined using Colby's formula (Colby, S.R. “Calculating synergistic and antagonistic responses of herbicide combinations”, Weeds, 15, 20-22, 1967) and compared with the observed efficacies. Colby's formula:
E=x+y−x·y/100
E expected efficacy, expressed in % of the untreated control, when using the mixture of the active compounds A and B at the concentrations a and b
x efficacy, expressed in % of the untreated control, when using the active compound A at the concentration a
y efficacy, expressed in % of the untreated control, when using the active compound B at the concentration b

The comparative compounds used were the compounds A and B known from the mixtures described in EP-A 988 790:

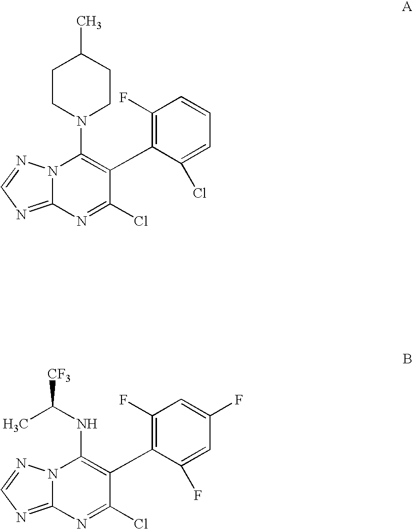

TABLE A
Individual active compounds

| | | Concentration of | Efficacy in |
|---|---|---|---|
| | | active compound in | % of the |
| | | the spray liquor | untreated |
| Example | Active compound | [ppm] | control |
| 1 | control | — | (90% |
| | (untreated) | | infection) |
| 2 | I | 4 | 56 |
| 3 | II | 16 | 0 |
| | (cyproconazole) | 4 | 0 |
| | | 1 | 0 |
| 4 | comparative | 4 | 11 |
| | compound A | | |
| 5 | comparative | 4 | 11 |
| | compound B | | |

TABLE B
Mixtures according to the invention

| | Mixture of active compounds | | |
|---|---|---|---|
| | Concentration | Observed | Calculated |
| Example | Mixing ratio | efficacy | efficacy*) |
| 6 | I + II | 78 | 56 |
| | 4 + 1 ppm | | |
| | 4:1 | | |
| 7 | I + II | 89 | 56 |
| | 4 + 4 ppm | | |
| | 1:1 | | |
| 8 | I + II | 94 | 56 |
| | 4 + 16 ppm | | |
| | 1:4 | | |

*)efficacy calculated using Colby's formula

TABLE C
Comparative tests

| | Mixture of active compounds | | |
|---|---|---|---|
| | Concentration | Observed | Calculated |
| Example | Mixing ratio | efficacy | efficacy*) |
| 9 | A + II | 22 | 22 |
| | 4 + 1 ppm | | |
| | 4:1 | | |
| 10 | A + II | 33 | 22 |
| | 4 + 4 ppm | | |
| | 1:1 | | |
| 11 | A + II | 44 | 22 |
| | 4 + 16 ppm | | |
| | 1:4 | | |
| 12 | B + II | 22 | 11 |
| | 4 + 1 ppm | | |
| | 4:1 | | |
| 13 | B + II | 22 | 11 |
| | 4 + 4 ppm | | |
| | 1:1 | | |
| 14 | B + II | 33 | 11 |
| | 4 + 16 ppm | | |
| | 1:4 | | |

*)efficacy calculated using Colby's formula

The test results show that, owing to strong synergism, the efficacy of the mixtures according to the invention against Plasmopara viticola is considerably higher than the cypronazole mixtures, proposed in EP-A 988 780, of the comparative compounds.

1. A fungicidial mixture comprising, as active components,
the triazolopyrimidine derivative of the formula I 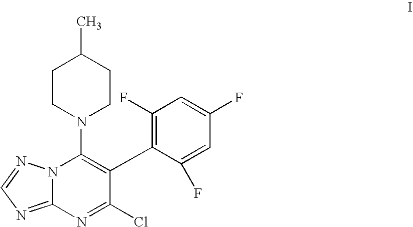
and
2) cyproconazole of the formula II,
in a synergistically effective amount.

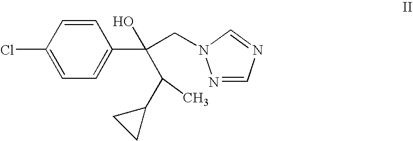

2. A fungicidal mixture comprising the compound of the formula I and the compound of the formula II in a weight ratio of from 100:1 to 1:100.

3. A composition comprising a liquid or solid carrier and a mixture according to claim 1 or 2.

4. A method for controlling harmful fungi from the class of the Oomycetes, which comprises treating the fungi, their habitat or the seed, the soil or the plants to be protected against fungal attack with an effective amount of the compound I and the compound II according to claim 1.

5. The method according to claim 4, wherein the compounds I and II are applied simultaneously, that is jointly or separately, or in succession.

6. The method according to claim 4, wherein the mixture is applied to the soil or the plants to be protected against fungal attack in an amount of from 5 g/ha to 1000 g/ha.

7. The method according to claim 4, wherein the mixture is applied in an amount of from 1 to 1000 g/100 kg of seed.

8. The method according to claim 4, wherein the harmful fungus Plasmopara viticola is controlled.

9. Seed comprising the mixture according to claim 1 or 2 in an amount of from 1 to 1000 g/100 kg.

10. The use of the compound II according to claim 1 for preparing a composition suitable for controlling Oomycetes.
